(19) United States
(12) Patent Application Publication
(10) Pub. No.: US 2009/0234161 A1
(43) Pub. Date: Sep. 17, 2009

(54) METHOD FOR THE PRODUCTION OF (METH)ACRYLIC ANHYDRIDE

(75) Inventor: Jean-Michel Paul, Metz (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Puteaux (FR)

(21) Appl. No.: 11/718,045

(22) PCT Filed: Oct. 18, 2005
(86) PCT No.: PCT/FR05/02573

§ 371 Date: Apr. 26, 2007

(30) Foreign Application Priority Data
Oct. 26, 2004 (FR) 0411396

(51) Int. Cl. C07C 57/00

(52) U.S. Cl. 562/887

(57) ABSTRACT

The present invention relates to an improved method for manufacturing (meth)acrylic anhydride by transanhydrization between (meth)acrylic acid and acetic anhydride, in the presence of air and in the presence of at least one polymerization inhibitor, characterized in that the polymerization inhibitor is chosen from the group formed from (a) metal salts of thiocarbamic or dithiocarbamic acid and their mixtures with a phenolic derivative or phenothiazine and its derivatives, and (b) N-oxyl compounds as a mixture with 2,6-di-tert-butyl-4-methylphenol taken alone or in the presence of 2,4-dimethyl-6-tert-butylphenol.

The present invention relates to an improved method for manufacturing (meth)acrylic anhydride by transanhydrization between (meth)acrylic acid and acetic anhydride.

The term “(meth)acrylic anhydride”, which will subsequently be denoted by (M)AA₂O, is understood to mean methacrylic anhydride MAA₂O or acrylic anhydride AA₂O.

The synthesis of (meth)acrylic anhydride by transanhydrization between (meth)acrylic acid and acetic anhydride has been the subject of numerous patents. Mention may more particularly be made of Application FR 2 592 040 that describes such a method in the absence of a catalyst and in the presence of a polymerization inhibitor. The implementation of this method however comes up against polymerization problems, one of the main difficulties lying in the choice of polymerization inhibitors. Indeed, it is well known that one of the tricky aspects of manufacturing and/or purifying (meth)acrylic monomers stems from the fact that these compounds are unstable and have a tendency to form polymers easily. This rending, caused by a radical reaction due to the effect of temperature, is particularly favoured during the steps of synthesizing and purifying these monomers, for example in the distillation steps. This results in the formation, in installation equipment, of solid polymer deposits that end up causing blockages and make it necessary to shut down the plant in order for cleaning to take place.

The polymerization inhibitors recommended in FR 2 592 040 for stabilizing the reaction medium are phenothiazine (PTZ), hydroquinone (HQ), methylene blue, iron sulphate, copper acetate or copper sulphate.

Patent Application US 2002/0161260, relating to a method for manufacturing unsaturated carboxylic acid anhydrides by transanhydrization in the presence of a catalyst, itself recommends the use of polymerization inhibitors such as hydroquinone (HQ), hydroquinone methyl ether (HQME), phenothiazine (PTZ), 2,4-dimethyl-6-tert-butylphenol (TOPANOL®A), 2,6-di-tert-butyl-4-methylphenol (TOPANOL®O or BHT), IRGANOX® 1010 (sold by Ciba AG Corporation), N,N′-diphenyl-p-phenylenediamine, or mixtures thereof.

In EP 1 273 565, TOPANOL®A and BHT, taken alone or as a mixture, are chosen in order to prevent the risk of polymerization in the reactor and in the distillation column during the synthesis and purification of (meth)acrylic anhydride.

Under these common stabilization conditions, during the synthesis of (meth)acrylic anhydride, despite everything the presence of a fine deposit of white powder is observed, which clogs up the walls, the bottom, the stirring shaft and the counterblades of the reactor. In addition, this white powder blocks the filters placed upstream of the container for storing the crude reaction product. This blocking problem requires the filters to be regularly cleaned, which is not easy with such lachrymatory products as methacrylic anhydride and especially acrylic anhydride.

Furthermore, polymerization inhibitors are known that are effective for stabilizing (meth)acrylic acids. For example, mention may be made of N-oxyl compounds in combination with a phenolic compound and phenothiazine (EP 620 206), or in combination with a manganese salt or a copper salt (EP 685 447) or else in combination with a phosphine derivative or a cobalt salt (EP 810 196). According to the Application GB 2 285 983, the polymerization during the distillation of vinyl compounds is inhibited using a copper dithiocarbamate compound in the presence of a metal such as chromium, manganese, titanium or cobalt and in the presence of phenothiazine, hydroquinone, p-methoxyphenol, cresol, phenol, tert-butylcatechol, diphenylamine or methylene blue.

It is not demonstrated whether these various systems, which one effective for (meth)acrylic acids, are effective in the presence of (meth)acrylic anhydrides.

Indeed, some of these stabilizers may be esterified by the anhydrides and therefore consumed in the reaction medium. It has been shown that under these conditions they lose their effectiveness. This is especially the case for compounds such as hydroquinone (HQ) or hydroquinone methyl ether (HQME).

The difficulty in choosing a polymerization inhibitor results from the fact that (meth)acrylic anhydrides and (meth)acrylic acids are not sensitive to the same polymerization inhibitors.

During the transanhydrization reaction between the (meth)acrylic acid and the acetic anhydride, the reaction medium contains the following species:

(meth)acrylic acid;

acetic anhydride;

(meth)acrylic anhydride;

mixed (meth)acrylic/acetic anhydride; and

acetic acid.

It is therefore important, in order to ensure good stabilization of the reaction medium, to use a polymerization inhibitor, or a mixture of polymerization inhibitors, capable of stabilizing all the polymerizable monomers present in the medium, more particularly both the (meth)acrylic and mixed anhydrides, and also the (meth)acrylic acids.

The Applicant company has therefore sought a polymerization inhibitor system capable of stabilizing both (meth)acrylic anhydrides and (meth)acrylic acids, in order to solve the clogging and filter-blocking problems during the manufacture of (meth)acrylic anhydride by transanhydrization.

The subject of the present invention is therefore an improved method for manufacturing (meth)acrylic anhydride by transanhydrization between (meth)acrylic acid and acetic anhydride, in the presence of air and in the presence of at least one polymerization inhibitor, characterized in that the polymerization inhibitor is chosen from the group formed from (a) metal salts of thiocarbamic or dithiocarbamic acid and their mixtures with a phenolic derivative or phenothiazine and its derivatives, and (b) N-oxyl compounds as a mixture with 2,6-di-tert-butyl-4-methylphenol taken alone or in the presence of 2,4-dimethyl-6-tert-butylphenol.

These novel stabilizing systems have, surprisingly, a remarkable effect and their use in the presence of air makes it possible to remove the clogging deposit of white powder as described previously, not only during the manufacture of (meth)acrylic anhydride, but also during its purification, its storage and its transport.

The invention also relates to the use of at least one polymerization inhibitor chosen from the group formed from (a) metal salts of thiocarbamic or dithiocarbamic acid and their mixtures with a phenolic derivative or phenothiazine and its derivatives, and (b) N-oxyl compounds as a mixture with 2,6-di-tert-butyl-4-methylphenol taken alone or in the presence of 2,6-di-tert-butyl-4-methylphenol and 2,4-dimethyl-6-tert-butylphenol, for manufacturing, purifying, storing or transporting (meth)acrylic anhydride.

The metal salts of thiocarbamic or dithiocarbamic acid are preferably chosen from copper dialkyl-dithiocarbamates in which the linear or branched alkyl groups, being identical or different, have a number of carbon atoms that ranges from 1 to 8. More particularly, copper diethyldithiocarbamate or copper dibutyldithiocarbamate will be used.

The phenolic derivatives are chosen from hindered phenolic derivatives, that is to say from phenolic derivatives substituted by linear or branched alkyl radicals, being identical or different, having a number of carbon atoms that ranges from 1 to 8. Preferably, 2,6-di-tert-butyl-4-methylphenol (BHT) or 2,4-dimethyl-6-tert-butylphenol (TOPANOL®A) will be used.

Phenothiazine and its derivatives such as methylene blue may be used in combination with a metal salt of thiocarbamic or dithiocarbamic acid.

By way of illustration of N-oxyl compounds that can be used according to the invention, mention may be made of the derivatives of 2,2,6,6-tetramethyl-1-piperidinyloxy (commonly known as TEMPO), especially 4-hydroxy-2,2,6,6-tetramethyl-1-piperidinyloxy (4-hydroxy-TEMPO), 4-methoxy-2,2,6,6-tetramethyl-1-piperidinyloxy (4-methoxy-TEMPO), 4-oxo-2,2,6,6-tetramethyl-1-piperidinyloxy (4-oxo-TEMPO) or 4-amino-2,2,6,6-tetra-methyl-1-piperidinyloxy (4-amino-TEMPO).

According to the invention, the N-oxyl compounds are used as a mixture with 2,6-di-tert-butyl-4-methylphenol (BHT) taken alone or in the presence of 2,4-dimethyl-6-tert-butylphenol (TOPANOL®A).

According to one particular embodiment of the invention, a mixture of 4-hydroxy-TEMPO with BHT or a mixture of 4-hydroxy-TEMPO with BHT and TOPANOL®A is used.

The amount of inhibitors, taken alone or as mixtures, used in the reactor is between 50 and 5000 ppm, more particularly between 300 and 4000 ppm and even more particularly between 1000 and 3000 ppm relative to the total weight of reactants used.

The reaction may be carried out in a reactor topped with a distillation column. Then, a double stabilization is preferably carried out, by introducing at least one polymerization inhibitor into the reactor and at least one polymerization inhibitor into the distillation column. Thus, any risk of polymerization in the reactor and in the column is avoided. The reactor inhibitor is preferably introduced into the initial charge of reactants. The distillation column inhibitor is preferably introduced into the distillation column, at the top of the column under reflux, throughout the synthesis, as a solution having a concentration between 0.2 and 6% in (meth)acrylic acid, acetic anhydride, acetic acid or (meth)acrylic anhydride, particularly as a 5% solution in acetic acid. The flow rate for introducing the inhibitor into the column is adjusted so as to have 2000 to 4000 ppm of inhibitor in the final product of the reactor.

In general, the reactor is stirred and heated by circulation of heat transfer fluid in a jacket or by recirculation through an external heat exchanger. The distillation column preferably has an efficiency greater than 10 theoretical plates. The column packing may be a conventional random or structured packing or a mixture of these two types of packing. The reaction temperature is generally between 50 and 120° C., preferably between 85 and 105° C. The pressure is adjusted depending on the reaction temperature chosen. In general, it is between 20 and 200 mmHg. The reaction may be carried out in isobaric mode, that is to say by setting the pressure and by changing the temperature up to a limiting value preferably set between 85 and 105° C., or in isothermal mode, namely by setting the temperature and by adjusting the pressure in the installation throughout the reaction in order to maintain this temperature. The column head temperature is advantageously adjusted during the reaction, depending on the pressure, so as to correspond to the distillation temperature of the acetic acid. By proceeding in this way, a top fraction containing more than 99% of acetic acid is obtained.

Air sparging is carried out during the whole reaction.

According to one preferred embodiment of the invention, the acetic acid produced by the reaction is distilled gradually as it is formed in order to displace the thermodynamic equilibria. According to another preferred embodiment of the invention, the acetic acid removed is at least partially replaced by introducing acetic anhydride and/or (meth)acrylic acid into the reaction medium during the reaction.

The crude product obtained may undergo a subsequent distillation step, if necessary after removal of the top fraction, on a distillation column, or using a short-residence-time apparatus such as a film evaporator.

### EXAMPLES

The following examples illustrate the present invention without however limiting scopethereof. The percentages therein are expressed as weight percentages.

The following abbreviations are used therein:

MAA: (meth)acrylic acid;

AA: acrylic acid;

MAA₂O: methacrylic anhydride;

AA₂O: acrylic anhydride;

Ac₂O: acetic anhydride;

AcOH: acetic acid;

Mixed: mixed acrylic/acetic anhydride or methacrylic/acetic anhydride depending on the case;

CB: copper dibutyldithiocarbamate;

BHT: 2,6-di-tert-butyl-4-methylphenol;

PTZ: phenothiazine;

HQ: hydroquinone;

HQME: hydroquinone methyl ether;

TOPANOL®A: 2,4-dimethyl-6-tert-butylphenol; and

OHT: 4-hydroxy-TEMPO: 2,2,6,6-tetramethylpiperidine-N-oxyl.

### Examples 1 to 9

255 g (2.5 mol) of Ac₂O, 344.9 g (4.01 mol) of MAA and one or more of the polymerization inhibitors listed in Table 1 were introduced into a mechanically stirred (anchor-type stirrer) glass jacketed reactor supplied with oil at 125° C., topped with a distillation column having a Multiknit Sulzer structured packing and an efficiency corresponding to 12 theoretical plates, with a condenser, reflux head, vacuum separator, receiver and traps, the assembly being able to operate under vacuum.

Air sparging (0.2 l/h) was maintained in the reaction medium throughout the duration of the synthesis.

During the reaction phase, the acetic acid produced by the reaction was distilled gradually as it formed in order to displace the thermodynamic equilibria towards the formation of MAA₂O.

The reaction temperature was maintained at 93° C. by gradually lowering the operating pressure from 140 to 18 mmHg.

Thus, 230 ml of a first fraction containing 99% of AcOH and 0.9% of Ac₂O were recovered. The excess Ac₂O, the mixed anhydride and the remainder of AcOH formed were then removed by distillation under 16 mmHg.

The crude product contained in the reactor that comprised 95% of MAA₂formed was then cooled to room temperature and filtered. It was stored in order to optionally be distilled so as to obtain a MAA₂having a purity greater than 99%. The installation used for synthesizing the crude product may be used for carrying out the distillation.

Examination of the cleanliness of the reactor and its attachments gave information on the effectiveness of the inhibitors used.

The inhibitor concentrations in Table 1 are expressed in parts per million (ppm) relative to the total charge of reactants introduced into the reactor. The low OHT concentrations enable colouring problems to be avoided.

TABLE 1

| Ex- | | | |
|---|---|---|---|
| ample | | Conc. | |
| No. | Inhibitor | (ppm) | Observations |
| 1 | TOPANOL ®A | 1000 | Lots of clogging on the reactor |
| | | | walls and dome. Cloudy crude |
| | | | product. Precipitation of solid |
| | | | in the crude product at low |
| | | | temperature after filtration. |
| 2 | BHT | 1000 | Same conclusion as in Example 1. |
| 3 | TOPANOL ®A | 1000 | Same conclusion as in Example 1. |
| | PTZ | 200 | |
| 4 | BHT | 1000 | Same conclusion as in Example 1. |
| | PTZ | 200 | |
| 5 | TOPANOL ®A | 1000 | Reduced clogging of the walls. |
| | BHT | 1000 | Precipitation of a smaller amount |
| | HQME | 8000 | of solid than in Example 1, at |
| | | | low temperature after filtration. |
| 6 | TOPANOL ®A | 1000 | Clogging of the reactor walls. |
| | OHT | 200 | Precipitation of solids in the |
| | | | cold crude product after filtration. |
| 7 | BHT | 1000 | Reactor perfectly clean. Little |
| | OHT | 200 | precipitate in the crude product |
| | | | at low temperature after filtration. |
| 8 | BHT | 1000 | Reactor perfectly clean. No |
| | TOPANOL ®A | 1000 | precipitate in the cold crude |
| | OHT | 200 | product after filtration. |
| 9 | CB | 2000 | Reactor perfectly clean. No |
| | | | precipitate in the cold crude |
| | | | product after filtration but |
| | | | crude product is slightly coloured. |

### Examples 10 to 12

The previously described installation was charged with 408 g (4 mol) of Ac₂O, 403.2 g (5.6 mol) of AA and one or more polymerization inhibitors listed in Table 2.

The reaction temperature was held around 90° C. by gradually lowering the operating pressure in the installation from 160 to 18 mmHg.

Air sparging (0.2 l/h) was maintained in the reaction medium throughout the duration of the synthesis.

During the reaction phase, the acetic acid produced by the reaction was distilled gradually as it formed in order to displace the thermodynamic equilibria towards the formation of AA₂O.

After removing the excess Ac₂O by vacuum distillation, the pure AA₂O was distilled under 18 mmHg.

Examination of the cleanliness of the reactor and its attachments after distillation of the AA₂O gave information on the effectiveness of the inhibitors used.

The inhibitor concentrations are expressed in parts per million (ppm) relative to the total charge of reactants introduced into the reactor.

TABLE 2

| Ex- | | | |
|---|---|---|---|
| ample | | Conc. | |
| No. | Inhibitor | (ppm) | Observations |
| 10 | CB | 2000 | Reactor clean after distillation of |
| | | | the AA₂O. Liquid residue. |
| 11 | CB | 250 | Same conclusion as in |
| | PTZ | 1000 | Example 10. |
| 12 | CB | 500 | Same conclusion as in |
| | BHT | 1000 | Example 10. |
| 13 | TOPANOL ®A | 1000 | Slight clogging of the reactor |
| | BHT | 1000 | walls. |
| | OHT | 200 | |

1. Method for manufacturing (meth)acrylic anhydrides comprising the step of transanhydrization between (meth)acrylic acid and acetic anhydride, in the presence of air and in the presence of at least one polymerization inhibitor, wherein the polymerization inhibitor is selected from the group consisting of (a) metal salts of thiocarbamic or dithiocarbamic acid and their mixtures with a phenolic derivative or phenothiazine and its derivatives, and (b) N-oxyl compounds as a mixture with 2,6-di-tert-butyl-4-methylphenol taken alone or in the presence of 2,4-dimethyl-6-tert-butylphenol.

2. Method according to claim 1, wherein the metal salt of dithiocarbamic acid is a copper dialkyldithiocarbamate having linear or branched alkyl groups, being identical or different, each with a number of carbon atoms that ranges from 1 to 8.

3. Method according to claim 1, wherein said metal salts of thuocarbamic or dithiocarbamic acid is copper diethyldithiocarbamate or copper dibutyldithiocarbamate.

4. Method according to claim 1, wherein the phenolic derivative is 2,6-di-tert-butyl-4-methylphenol or 2,4-dimethyl-6tert-butylphenol.

5. Method according to claim 1, wherein the N-oxyl compound is a derivative of 2,2,6,6-tetramethyl-1-piperidinyloxy (TEMPO).

6. Method according to claim 5, wherein the N-oxyl compound is 4-hydroxy-TEMPO, 4-methoxy-TEMPO, 4-oxo-TEMPO, or 4-amino-TEMPO.

7. Method according to claim 5, wherein the N-oxyl compound is 4-hydroxy-TEMPO.

8. Method according to claim 1, wherein the amount of inhibitors, taken alone or as mixtures, used in the reactor is between 50 and 5000 ppm, relative to the total weight of reactants used.

9. Method according to claim 1, wherein the reaction is carried out in a reactor topped with a distillation column.

10. Method according to claim 9, wherein at least one polymerization inhibitor is introduced into the reactor and at least one polymerization inhibitor is introduced into the distillation column.

11. Method according to claim 10, wherein the distillation column inhibitor is introduced into the distillation column throughout the synthesis, in solution, having a concentration between 0.2 and 6%, in (meth)acrylic acid, acetic anhydride, acetic acid or (meth)acrylic anhydride.

12. (canceled)

13. Method according to claim 8, wherein the amount of inhibitors, taken alone or as mixtures, used in the reactor is between 300 and 4000 ppm, relative to the total weight of reactants used.

14. Method according to claim 8, wherein the amount of inhibitors, taken alone or as mixtures, used in the reactor is between 1000 and 3000 ppm, relative to the total weight of reactants used.
